(19) United States
(12) Patent Application Publication
(10) Pub. No.: US 2022/0143350 A1
(43) Pub. Date: May 12, 2022

(54) SUCTION CATHETER ASSEMBLIES AND ASSEMBLIES INCLUDING A SUCTION CATHETER ASSEMBLY

(71) Applicant: SMITHS MEDICAL INTERNATIONAL LIMITED, Ashford (GB)

(72) Inventors: Steven Mark Tupper, Hythe (GB); Usamah Hanif, Ashford (GB)

(73) Assignee: SMITHS MEDICAL INTERNATIONAL LIMITED, Ashford (GB)

(21) Appl. No.: 17/432,554

(22) PCT Filed: Feb. 26, 2020
(86) PCT No.: PCT/GB2020/000022

§ 371 Date: Aug. 20, 2021

(30) Foreign Application Priority Data
Mar. 2, 2019 (GB) 1902868.7

(51) Int. Cl. A61M 16/08; A61M 16/04

(52) U.S. Cl. CPC A61M 16/0825; A61M 2240/00; A61M 16/0463

(57) ABSTRACT

A paediatric closed system suction catheter assembly has a patient end fitting (20) through which a suction catheter (24) is advanced into the tracheal tube (1). The patient end fitting comprises two parts: a machine end portion (30) including a wiper seal (32) and a ventilation side port (37), and a patient end portion (31) with a tapered spigot (42) inserted directly into the machine end (12) of the tube (1) without any intervening connector. The machine end and patient end portions (30) and (31) are connected together by a rotatable joint (43) formed by part-spherical surfaces (39) and (41) on the two portions.

This invention relates to suction catheter assemblies of the kind including a patient end fitting at one end adapted to be attached with the machine end of a tracheal tube, a machine end fitting at an opposite end, a suction catheter extending from the machine end fitting to the patient end fitting such that the patient end of the suction catheter can be extended through the patient end fitting, and a protective, flexible envelope extending around and along the suction catheter and attached to the machine end fitting at one end and to the patient end fitting at the other end.

During intubated ventilation of a patient secretions that collect in the respiratory passages or in the tracheal tube need to be removed periodically. A conventional suction catheter can be used for this purpose but it is usually preferred to use a closed system suction catheter assembly. A closed system suction assembly comprises a manifold at the patient end with a sliding, wiper seal through which a suction catheter can be advanced and withdrawn. A flexible envelope is joined at one end to the manifold and encloses the catheter along its length. The other end of the envelope and the catheter are joined with a machine end component including a suction control valve and a connector. The connector connects the catheter to a suction source and the valve enables the clinician to control the suction applied by the catheter. Examples of closed system suction catheters are described in U.S. Pat. Nos. 5,269,728, 5,300,043, 4,569,344, 4,638,539, 4,872,579, 5,167,622, 5,779,687, 5,325,850, 5,490,503, 5,419,769, 5,460,613, 5,349,950, GB2394761, GB2400160, U.S. Pat. Nos. 6,109,259, 6,227,197, EP801577B, WO96/09082, EP1239907B, EP1478424B, U.S. Pat. No. 6,588,427, EP1620148B, US2004/0221852, EP1911482A, EP1795217A, US2007/0282250, WO2007/143502, US2008/0188833, U.S. Pat. Nos. 6,227,200, 6,543,451, EP1239909B, U.S. Pat. No. 6,602,219, EP1347798, WO02/49680, U.S. Pat. No. 6,609,520, WO/055143, U.S. Pat. Nos. 6,805,125, 6,923,184, 7,021,313, 7,191,782, WO2004/101045, U.S. Pat. No. 7,263,997, WO2004/103448, WO00/15276, WO11/020985, EP637257B, EP1113835B, EP1210957A, EP1237612B, U.S. Pat. No. 7,152,603, EP1267957B, U.S. Pat. No. 6,978,783, US2004/0007236, US2005/0211253, US2005/0211245, US2005/0235987, U.S. Pat. No. 7,059,322, WO2004/032817, US2008/0135051, U.S. Pat. Nos. 4,836,199, 4,850,350, 4,967,743, 5,025,806, 5,083,561, 5,220,916, 5,215,522, 5,255,676, 5,277,177, 5,309,902, 5,333,606, 5,343,857, 5,487,381, 5,513,628, 5,791,337, EP1343552A, WO02/49699, U.S. Pat. No. 6,612,304, EP1322371A, WO02/28463, U.S. Pat. No. 6,629,530, WO02/051485, U.S. Pat. No. 6,769,430, EP1330284, WO02/36191, U.S. Pat. No. 6,886,561, WO2004/034946, U.S. Pat. No. 7,188,623, WO2006/014431, U.S. Pat. No. 7,341,059, WO2005/094925, WO2006/103233, WO2007/030388, WO2009/003135, U.S. Pat. Nos. 4,838,255, 5,107,829, 5,133,345, 5,642,726, 6,702,789, 7,458,955, 7,273,473, 5,139,018, 4,327,723, 4,515,592, 6,099,519, EP695556B, U.S. Pat. Nos. 5,065,754, 5,730,123, 5,207,220, 5,309,903 and 7,086,402. Closed system suction catheters are available from various manufacturers including Smiths Medical, Kimberley Clark, Covidien and Viasys.

In neonatal/paediatric suction catheter assemblies the patient end of the assembly is provided by a fixed male tapered surface for making a sealing connection in the standard female 15 mm or 8 mm connector fixed to the machine end of the tracheal tube. The problem with this fixed connection is that manipulation of the assembly during the suctioning procedure can apply torsion to and cause movement of the tracheal tube. This is a particular problem with neonatal and paediatric patients because it may cause irritation to the delicate airway tissue. It is also particularly important with neonatal and paediatric suction catheter assemblies to minimise any increase in deadspace within the assembly since this can adversely affect gas transfer with the patient.

It is an object of the present invention to provide an alternative suction catheter assembly and an assembly including a suction catheter assembly.

According to one aspect of the present invention there is provided a closed system suction catheter assembly of the above-specified kind, characterised in that the patient end fitting includes a forward portion shaped for insertion directly in the machine end of a shaft of the tracheal tube without an intervening connector, and that the patient end fitting includes a machine end portion to which the flexible envelope is attached and a rotatable joint between the forward and machine end portions to allow for relative angular rotation between the machine end portion and the forward portion.

The forward portion of the patient end fitting is preferably tapered to an increasing diameter rearwardly along its length.

According to another aspect of the present invention there is provided an assembly of a tracheal tube and a closed system suction catheter assembly, the tracheal tube having a shaft of a flexible plastics material with a patient end and a machine end, the suction catheter assembly including a patient end fitting at one end, a machine end fitting at an opposite end, and a suction catheter extending from the machine end fitting to the patient end fitting such that the patient end of the suction catheter can be extended through the patient end fitting, and a protective, flexible envelope extending around and along the suction catheter and attached to the machine end fitting at one end and to the patient end fitting at the other end, characterised in that the patient end fitting includes a tapered forward portion inserted directly in the machine end of the shaft of the tracheal tube without an intervening connector, and that the patient end fitting includes a machine end portion to which the flexible envelope is attached and a rotatable joint between the forward and machine end portions to allow for relative angular rotation between the machine end portion and the forward portion.

The rotatable joint may include a part-spherical surface on one part fitted within a part-spherical surface on another part. The one part may be on the machine end portion and the other part being on the forward portion. The machine end portion of the patient end fitting preferably includes a wiper seal through which the catheter extends. The patient end fitting preferably includes a side port opening into the fitting. The side port may be provided on the machine end portion and be formed with a female tapered coupling adapted to receive a cooperating male coupling connected with a ventilation circuit. The forward portion of the patient end fitting is preferably adapted to extend along substantially its entire length in the machine end of the shaft of the tracheal tube. The forward portion of the patient end fitting may be bonded inside the tube. The tracheal tube may be a tracheostomy tube and may be a paediatric tube.

A closed system suction catheter assembly and an assembly of a tracheal tube and a suction catheter assembly, according to the present invention, will now be described, by way of example, with reference to the accompanying drawings, in which:

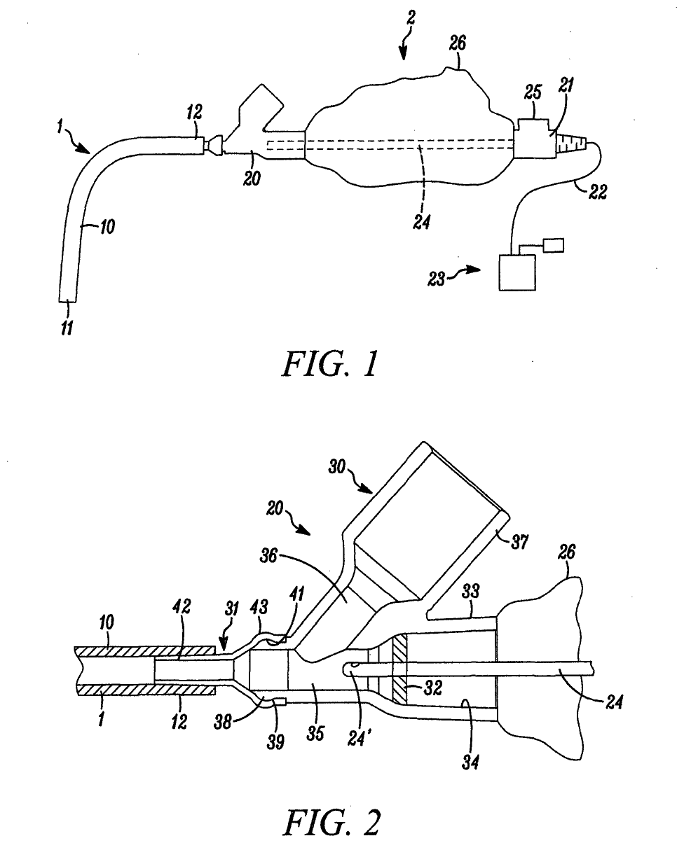

FIG. 1 is a side elevation view of the assembly of the tracheal tube and the suction catheter assembly;

FIG. 2 is an enlarged, sectional side elevation view of a first form of patient end fitting of the suction catheter assembly;

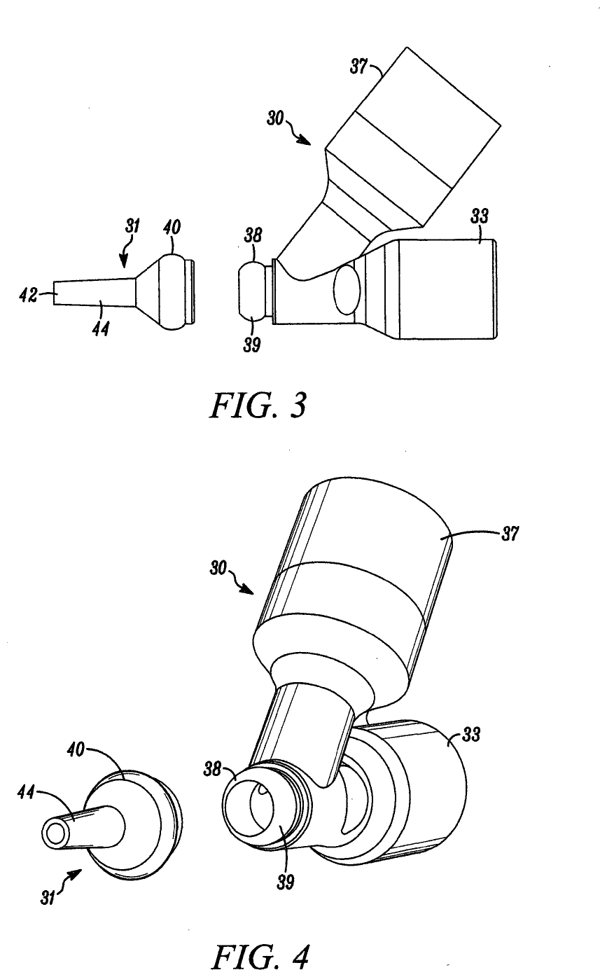

FIG. 3 is an enlarged side elevation view of the fitting in FIG. 2 with the forward portion separate from the rest of the fitting;

FIG. 4 is a perspective view of the fitting shown in FIG. 2 with the forward portion separated from the rest of the fitting; and

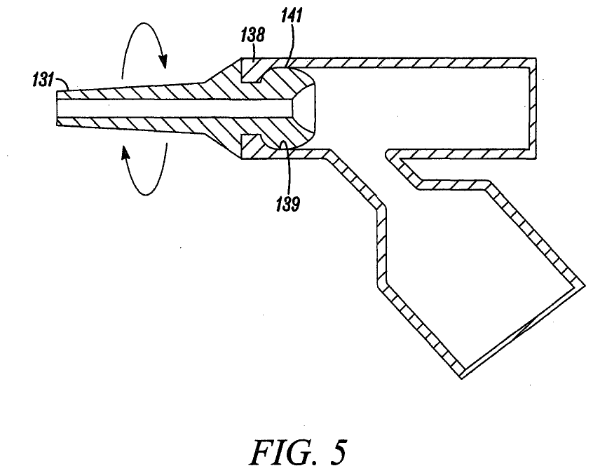

FIG. 5 is an enlarged, sectional side elevation view of an alternative, modified form of patient end fitting.

With reference first to FIG. 1, there is shown a paediatric tracheostomy tube 1 having a curved shaft 10 of a flexible plastics material with a patient end 11 adapted for location within the trachea and a machine end 12 adapted to project externally of the patient through a tracheostomy formed though neck tissue. A suction catheter assembly 2 has a patient end fitting 20 fitted directly into the machine end 12 of the shaft 10 of the tube 1 without the intervention of any connector. A machine end fitting 21 at the opposite end of the assembly 2 is connected via suction tubing 22 to a suction source 23.

The suction catheter assembly 2 includes a suction catheter 24 fixed at its machine end with the machine end fitting 21, which includes a conventional manually-operable valve 25 that can be used to control connection of the bore of the suction catheter to the suction source 23 and hence control the application of suction to the catheter. A flexible outer sleeve or envelope 26 encloses the catheter 24 along its length and is connected at opposite ends to the patient end fitting 20 and the machine end fitting 21 respectively.

The patient end fitting 20 is shown in more detail in FIGS. 2 to 4. The fitting 20 comprises two moulded plastics parts, namely a main, machine end body portion 30 and a separately-formed forward portion 31. The fitting also includes a wiper seal 32 housed in the main body portion 30. The main body portion 30 provides a Y-shape manifold with an axially-aligned cylindrical collar 33 to which the patient end of the envelope 26 is attached and through which the suction catheter 24 extends. The wiper seal 32 is mounted in the collar 33 towards the patient end of its bore 34. The bore 34 reduces in diameter on the patient side of the wiper seal 32 and opens into a central cavity 35. A side passage 36 also opens into the central cavity 35 from a side port or female tapered coupling 37 shaped to receive within it a conventional 15 mm or 8 mm male tapered coupling (not shown) connected with a ventilation circuit. Alternatively, the side port 37 can be left open to atmosphere when the patient is breathing spontaneously. The side port 37 is inclined at an angle of about 45° relative to the axis of the fitting 20. The main body portion 30 is terminated at its patient end by a short axial stem 38 forming internally a continuation of the cavity 35 and is aligned with the bore 34. On its outer surface the stem 38 is formed with one half of a rotatable coupling 43 provided by a part-spherical surface 39 extending around an angle θ of about 30°.

The other part of the patient end fitting 20, that is, the forward portion or adaptor 31 is moulded separately from the main body portion 30 but is assembled onto this. The adaptor 31 has an enlarged rear or machine end 40 shaped on its inner surface with a part-spherical surface 41 of the same size and shape as the surface 39 on the outside of the stem 38. The nature of the plastics material from which the body portion 30 and adaptor 31 are moulded provides sufficient resilience to allow the rear end 40 of the adaptor to be snapped onto the outside of stem and provide a secure rotatable joint or coupling 43 between the two components. The opposite end of the adaptor 31 is formed by a forward portion or spigot 42 with an external surface 44 that tapers along its length to an increased diameter rearwardly. The dimensions of the spigot 42 are selected such that it can be inserted directly in the machine end 12 of the shaft 10 of the tracheostomy tube 1 and form a secure connection with the shaft by virtue of the resilience of the shaft material. The length of the spigot 42 is chosen so that the machine end 12 of the shaft 10 extends along substantially the entire length of the spigot, thereby minimising the extra deadspace contributed by the patient end fitting 20. The adaptor 31 could be bonded to the tube 1 by applying a solvent or adhesive to the outer, tapered surface 43 of the spigot 42. This bonding could be carried out by the clinician or in the factory so that the tube 1 and suction catheter assembly 2 are supplied already joined with one another.

In normal use, the suction catheter assembly 2 is fitted with the tracheostomy tube 1 by the clinician and is left in place so that the patient can breathe through the side port 37 on the patient end fitting 20. The rotatable connection 43 between the adaptor 31 and the main body portion 30 enables the side port 37 to be rotated to the desired angle so that any connected ventilation tubing can be positioned where it causes minimum force on the tracheostomy tube 1 and hence minimum discomfort to the patient.

When suction is required, the clinician advances the suction catheter 24 forwardly, in a patient direction by manipulation through the envelope 26 so that its tip 24′ moves through the adaptor 31 into the tracheostomy tube 1. At the same time, the clinician actuates the suction control valve 25 so that suction is applied from the suction source 23 via the suction tubing 22 to the suction catheter 24. The rotatable coupling 43 also helps isolate the tracheostomy tube 1 from torque applied to the body portion 30 thereby enabling the suction catheter assembly 2 to be manipulated during suction, with minimum force being applied to the tracheostomy tube. When suction has been completed the suction valve 25 is released and allowed to closed, the suction catheter 24 being pulled rearwardly back into the patient end fitting 20 of the suction catheter assembly 2. The length of the envelope 26 and suction catheter 24 are selected so that, when the catheter is pulled rearwardly to its full extent, its patient end tip 24′ locates on the patient side of the wiper seal 32, with the shaft of the catheter extending through the opening of the wiper seal. Because one part of the rotatable coupling 43 is provided by the adaptor 31 that makes direct connection with the shaft 10 of the tracheal tube 1 the increase in deadspace contributed by the suction catheter assembly is minimised.

FIG. 5 shows a modified variant of the arrangement described above where the forward portion or adaptor 131 is formed at its machine end with an external part-spherical surface 141 that engages with a cooperating part-spherical surface 139 formed around the inside of the stem 138.

The invention could be used with other forms of tracheal tube such as endotracheal tubes and is not confined to paediatric use.

Various different types of rotatable coupling are possible including those with other than part-spherical surfaces and couplings that only enable rotation through a limited angle less than 360°.

1-12. (canceled)

13. A closed system suction catheter assembly including a patient end fitting at one end adapted to be attached with a machine end of a tracheal tube, a machine end fitting at an opposite end, a suction catheter extending from the machine end fitting to the patient end fitting such that the patient end of the suction catheter can be extended through the patient end fitting, and a protective, flexible envelope extending around and along the suction catheter and attached to the machine end fitting at one end and to the patient end fitting at the other end, characterised in that the patient end fitting includes a forward portion shaped for insertion directly in the machine end of a shaft of the tracheal tube without an intervening connector, and that the patient end fitting includes a machine end portion to which the flexible envelope is attached and a rotatable joint between the forward portion and the machine end portion to allow for relative angular rotation between the machine end portion and the forward portion.

14. An assembly according to claim 13, characterised in that the forward portion of the patient end fitting is tapered to an increased diameter rearwardly along its length.

15. An assembly according to claim 13, characterised in that the rotatable joint includes a part-spherical surface on one part fitted within a part-spherical surface on an other part.

16. An assembly according to claim 15, characterised in that the one part is on the machine end portion and that the other part is on the forward portion.

17. An assembly according to claim 13, characterised in that the machine end portion of the patient end fitting includes a wiper seal through which the catheter extends.

18. An assembly according to claim 13, characterised in that the patient end fitting includes a side port opening into the fitting.

19. An assembly according to claim 18, characterised in that the side port is provided on the machine end portion and is formed with a female tapered coupling adapted to receive a cooperating male coupling connected with a ventilation circuit.

20. An assembly according to claim 13, characterised in that the forward portion of the patient end fitting is adapted to extend along substantially its entire length in the machine end of the shaft of the tracheal tube.

21. An assembly according to claim 13, characterised in that the tracheal tube is a tracheostomy tube.

22. An assembly according to claim 13, characterised in that the tracheal tube is a paediatric tube.

23. An assembly of a tracheal tube and a closed system suction catheter assembly, the tracheal tube having a shaft of a flexible plastics material with a patient end and a machine end, the suction catheter assembly including a patient end fitting at one end, a machine end fitting at an opposite end, and a suction catheter extending from the machine end fitting to the patient end fitting such that the patient end of the suction catheter can be extended through the patient end fitting, and a protective, flexible envelope extending around and along the suction catheter and attached to the machine end fitting at one end and to the patient end fitting at another end, characterised in that the patient end fitting includes a tapered forward portion inserted directly in the machine end of the shaft of the tracheal tube without an intervening connector, and that the patient end fitting includes a machine end portion to which the flexible envelope is attached and a rotatable joint between the forward portion and the machine end portion to allow for relative angular rotation between the machine end portion and the forward portion.

24. An assembly according to claim 23, characterised in that the forward portion of the patient end fitting is bonded inside the tube.

25. An assembly according to claim 23, characterised in that the rotatable joint includes a part-spherical surface on one part fitted within a part-spherical surface on an other part.

26. An assembly according to claim 25, characterised in that the one part is on the machine end portion and that the other part is on the forward portion.

27. An assembly according to claim 23, characterised in that the machine end portion of the patient end fitting includes a wiper seal through which the catheter extends.

28. An assembly according to claim 23, characterised in that the patient end fitting includes a side port opening into the fitting.

29. An assembly according to claim 28, characterised in that the side port is provided on the machine end portion and is formed with a female tapered coupling adapted to receive a cooperating male coupling connected with a ventilation circuit.

30. An assembly according to claim 23, characterised in that the forward portion of the patient end fitting is adapted to extend along substantially its entire length in the machine end of the shaft of the tracheal tube.

31. An assembly according to claim 23, characterised in that the tracheal tube is a tracheostomy tube.

32. An assembly according to claim 23, characterised in that the tracheal tube is a paediatric tube.
